(19) United States
(12) Patent Application Publication
(10) Pub. No.: US 2010/0240048 A1
(43) Pub. Date: Sep. 23, 2010

(54) BIOLOGICAL SAMPLE REACTION CHIP, BIOLOGICAL SAMPLE CHARGING DEVICE, BIOLOGICAL SAMPLE QUANTIFYING DEVICE, AND BIOLOGICAL SAMPLE REACTION METHOD

(75) Inventor: Fumio TAKAGI, Chino (JP)

Correspondence Address: HARNESS, DICKEY & PIERCE, P.L.C., P.O. BOX 828, BLOOMFIELD HILLS, MI, 48303, US

(73) Assignee: SEIKO EPSON CORPORATION, Tokyo (JP)

(21) Appl. No.: 12/712,416

(22) Filed: Feb. 25, 2010

(30) Foreign Application Priority Data
Mar. 17, 2009 (JP) 2009-063967

(51) Int. Cl. C12Q 1/68; C12M 1/34

(52) U.S. Cl. 435/6; 435/287.2

(57) ABSTRACT

A biological sample reaction chip includes, a first path having a first width, a second path having a second width wider than the first width wherein the first path and the second path being connected, a first opening through which a first liquid is introduced into the first path, and a second opening through which a second liquid unmixable with the first liquid is introduced into the first path.

This application claims priority to Japanese Patent Application No. 2009-063967, filed Mar. 17, 2009, the entirety of which is hereby incorporated by reference.

### BACKGROUND

1. Technical Field

The present invention relates to a biological sample reaction chip, a biological sample charging device, a biological sample quantifying device, and a biological sample reaction method for nucleic acid amplification or the like.

2. Related Art

A method of performing chemical analysis, chemical synthesis, biological analysis and the like by using a micro fluid chip having a micro path on a glass substrate or the like has been attracting attention. The micro fluid chip is called the micro total analytical system (micro TAS), the lab-on-a-chip or the like as a method providing advantages such as smaller amounts of necessary samples and reagents, shorter reaction time, and a smaller volume of wastes than those of a related-art system. Thus, the micro fluid chip is expected to be used in many fields such as medical examination, environmental or food on-site analysis, and production of medical supplies, chemical supplies and the like. Since only a small amount of reagents are required, the cost for testing can be lowered. Also, since only small amounts of samples and reagents are needed, the reaction time can be considerably reduced and thus efficiency can be improved. Particularly when the micro fluid chip is used for the purpose of medical examination, the required quantity of blood or other specimens prepared as samples can be reduced. Therefore, the burden imposed on a patient can be lowered.

The polymerase chain reaction (PCR) is well known as a method for amplifying genes such as a DNA and an RNA used as a sample. In the PCR, a mixture of a target DNA as the amplification target (“target DNA”) and reagents are provided into a tube, and the mixture is reacted repeatedly under controlled temperature for a cycle of several minutes using a thermal cycler. The temperature needs to be controlled during each cycle, for example at 62° C., 72° C., and 95° C. in a three step reaction cycle, or 62° C. and 95° C. in a two step reaction cycle. According to this method, only the target DNA can be approximately doubled per one temperature cycle caused by an enzyme called polymerase.

Recently, a method called a real-time PCR using a particular fluorescent probe such as Taqman® and SYBRGreen© capable of quantifying target DNA as amplifying it. The real-time PCR is widely used for studies and clinical tests because of its high measurement sensitivity and reliability.

Quantifying DNA by the real-time PCR requires drawing an analytical curve showing the relationship between the initial amount of the target nucleic acid and the cycle number at which the fluorescent intensity has reached a certain level. The measurement result may deviate from the analytical curve due to possible existence of a substance in a specimen that may intervene with or prevent the amplification reaction. In this case, the reliability may be lowered.

According to the related-art system, the standard amount of the reaction liquid necessary for the PCR is several tens of microliters. Also, basically only a single type of gene can be measured by one reaction system. Adding a plurality of fluorescent probes having distinguishable different colors may enable to simultaneously measure four different types of genes, however, when a larger number of types of genes are to be simultaneously measured, the number of the reaction systems needs to be increased. The amount of the DNA extracted from a specimen is generally small, and reagents are costly. Thus, it makes it difficult to simultaneously measure a large number of reaction systems at once.

Miniaturizing a reaction chip may also be workable, however, dividing substantially an equal amount of specimen liquid accurately into each chip is difficult, and the overall amount of target nucleic acids contained in one reaction chip is so little that accurate quantification of the target DNA may be difficult.

JP-A-2001-269196 discloses a method called the critical dilution for measuring the target nucleic acids. The critical dilution performs PCR after gradually diluting specimen liquid and examines the concentration at which amplification of the target nucleic acid becomes unrecognizable to estimate the initial concentration of the target nucleic acids. Another known method is to perform PCR in plural reaction chips each containing specimen liquid diluted until the number of averaged target nucleic acids per chip becomes not more than 1 copy, and calculate the ratio of the reaction chips from which the target nucleic acid is detected to estimate the concentration based on Poisson distribution.

The critical dilution method, however, requires diluting specimen liquid in many steps to perform amplification reaction at each step for a specimen whose concentration is unknown. In this case, higher cost and longer time are needed.

### SUMMARY

It is an advantage of some aspects of the invention to provide a biological sample reaction chip, a biological sample charging device, a biological sample quantifying device, and a biological sample reaction method capable of efficiently quantifying nucleic acids by a critical dilution method using a small amount of reaction liquid.

A biological sample reaction chip according to a first aspect of the invention includes, a first path having a first width, a second path having a second width wider than the first width wherein the first path and the second path being connected, a first opening through which a first liquid is introduced into the first path, and a second opening through which a second liquid unmixable with the first liquid is introduced into the first path.

According to this structure, a plurality of the first liquid masses are separable by the second liquid unmixable with the first liquid in the first path. The second path is made wider than the first path so as to be able to form the first liquid masses different in volume from the first path. Thus, quantification of nucleic acids by the critical dilution method can be efficiently carried out. Since the liquid masses having relatively large volumes are supplied to the downstream part, or the second path, having a larger width than the upstream part, or the first path, the length in the longitudinal direction of each of the first liquid mass thus formed does not become too large in relation to the width. As a result, the target nucleic acids in the first liquid is distributed substantially equally in a single mass, not scarcely distributed in the longitudinal direction. Moreover, dividing a target biological sample into a tube is not necessary if using the chip of the invention. Even a very small amount of target biological sample that might be difficult to be distributed using a pipette or the like can be used for reaction.

A biological sample charging device according to a second aspect of the invention includes: a biological sample reaction chip which includes, a first path having a first width, a second path having a second width wider than the first width wherein the first path and the second path being connected, a first opening through which a first liquid is introduced into the first path, a second opening through which a second liquid unmixable with the first liquid is introduced into the first path, a first pump capable of supplying the first liquid into the biological sample reaction chip, and a second pump capable of supplying the second liquid into the biological sample reaction chip.

According to this structure, in addition to the above, a plurality of the first liquid masses are separable by the second liquid unmixable with the first liquid in the first path by supplying the first liquid and the second liquid into the chip using the two different pumps.

It is preferable that the biological sample charging device further includes a pump control unit to control a liquid feeding speed of either or both of the first pump and the second pump, or to stop either or both of the first pump or the second pump at a desired timing.

According to this structure, in addition to the above, controlling the movement of the first pump having the first liquid and the second pump having the second liquid realizes forming the first liquid masses in different volumes with a desired spacing between each of the first liquid masses.

A biological sample quantifying device according to a third aspect of the invention includes, a first path having a first width, a second path having a second width wider than the first width wherein the first path and the second path being connected, a first opening through which a first liquid is introduced into the first path, a second opening through which a second liquid unmixable with the first liquid is introduced into the first path, a first pump capable of supplying the first liquid into the biological sample reaction chip, a second pump capable of supplying the second liquid into the biological sample reaction chip, a biological sample reaction unit in which biological sample reaction occurs, and a detection unit which measures a result of a biological sample reaction.

According to this structure, in addition to the above, quantification of nucleic acids by the critical dilution can be efficiently carried out by measuring the result of the biological sample reaction occurred in each reaction chip. A biological sample charging method according to a fourth aspect of the invention includes, supplying a first liquid and a second liquid into a first path of a biological sample reaction to form a plurality of first liquid masses and a plurality of second liquid masses until either of the first or second liquid reaches at the end of a second path of the biological sample reaction chip, and controlling supplying movement of the first liquid or the second liquid being supplied so that the plurality of the first liquid masses and the plurality of the second liquid masses in the first path are smaller in volume than that in the second path.

By measuring the result of the biological sample reaction occurred inside the reaction chip, quantification of nucleic acids by the critical dilution can be efficiently carried out. Since the liquid masses having relatively large volumes are supplied to the downstream part (the second path), having a larger width than the upstreampart (the first path), the length in the longitudinal direction of each of the first liquid mass thus formed does not become too large in relation to the width. As a result, the target nucleic acids in the first liquid is distributed substantially equally in a single mass, not scarcely distributed in the longitudinal direction. Moreover, dividing a target biological sample into a tube is not necessary if using the chip of the invention. Even a very small amount of target biological sample that might be difficult to be distributed using a pipette or the like can be used for reaction.

### BRIEF DESCRIPTION OF THE DRAWINGS

The invention will be described with reference to the accompanying drawings, wherein like numbers reference like elements.

FIG. 1 schematically illustrates a general structure of a biological sample quantifying device including a biological sample reaction chip according to an embodiment of the invention.

FIG. 2A is a perspective view illustrating a general structure of the biological sample reaction chip according to the embodiment of the invention.

FIG. 2B is a cross-sectional view taken along a line B-B in FIG. 2A.

FIG. 3 illustrates charging of reaction liquid and mineral oil to the biological sample reaction chip according to the embodiment of the invention.

FIG. 4 illustrates charging of the reaction liquid and the mineral oil to the biological sample reaction chip according to the embodiment of the invention.

FIG. 5 illustrates another example of the biological sample reaction chip according to the invention.

### DESCRIPTION OF EXEMPLARY EMBODIMENT

An embodiment according to the invention is hereinafter described with reference to the drawings.

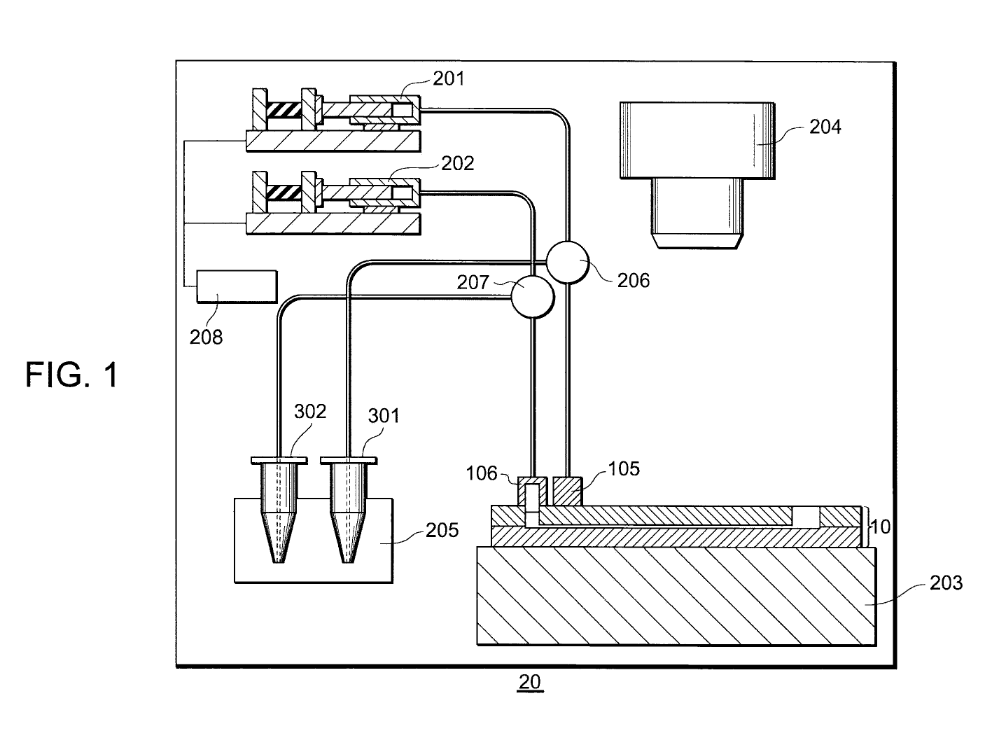

FIG. 1 schematically illustrates a general structure of a biological sample quantifying device 20 including a biological sample reaction chip (biological sample reaction chip) 10 according to this embodiment. The biological sample quantifying device 20 includes syringe pumps (first pump and second pump) 201 and 202, a temperature control heat block (biological sample reaction unit) 203, an optical detector (detection unit) 204, a reaction liquid storage unit 205, valves 206 and 207, and a pump control unit 208.

The syringe pumps 201 and 202 are connected with the pump control unit 208 such that each pump can be driven at an arbitrary liquid feeding speed.

The heat block 203 is a device for maintaining a predetermined temperature of the biological sample reaction chip 10 during PCR process (biological sample reaction process). The heat block 203 is connected with a controller (not shown) capable of controlling the setting temperature and time according to the temperature cycle of the PCR process.

The optical detector 204 may include a CCD camera or the like. The reaction liquid storage unit 205 accommodates chips 301 and 302 filled with reaction liquid or the like. The chips 301 and 302 are connected with the valves 206 and 207 and the syringe pumps 201 and 202 via silicon tubes or the like, respectively.

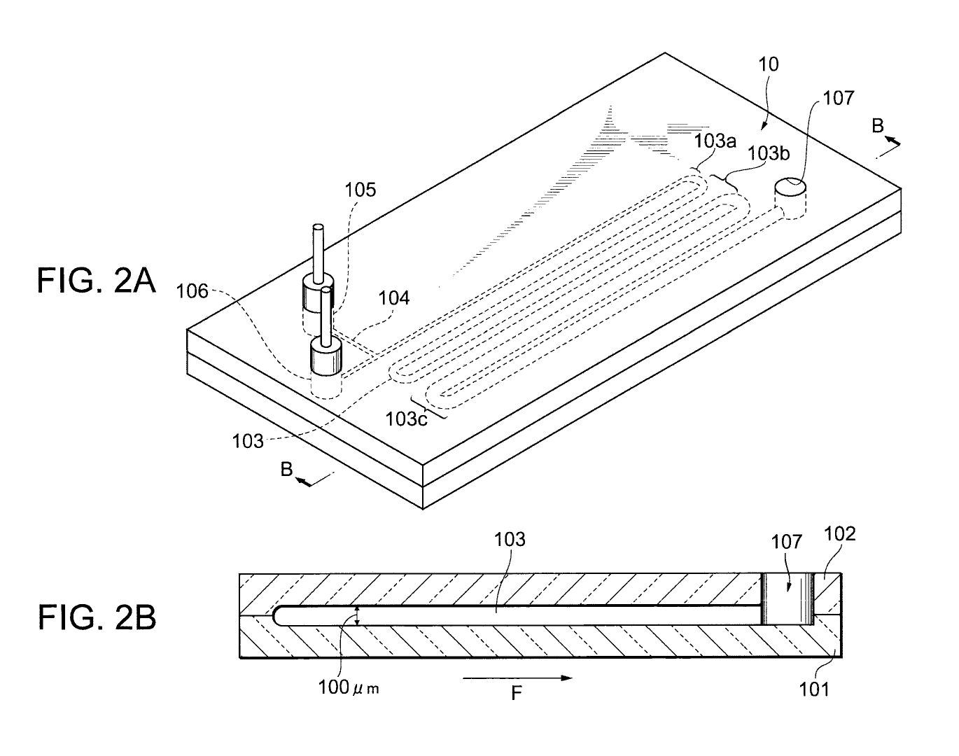

FIG. 2A is a perspective view schematically illustrating a general structure of the biological sample reaction chip 10 according to this embodiment of the invention. FIG. 2B is a cross-sectional view taken along a line B-B in FIG. 2A. As illustrated in the figures, the biological sample reaction chip 10 includes transparent substrates 101 and 102, a flow path 103 including a first flow path 103a, a second flow path 103b and a third flow path 103b, a feeding path 104, a first opening 105, a second opening 106, and a third opening 107.

As illustrated in FIGS. 2A and 2B, the biological sample reaction chip 10 has the two transparent substrates 101 and 102 affixed to each other. Each of the transparent substrates 101 and 102 has a groove constituting a part of the flow path 103 to form the three-dimensional flow path 103 when the transparent substrates 101 and 102 are affixed to each other. Each of the transparent substrates 101 and 102 further has a groove constituting a part of the flow path 104 to form the three-dimensional flow path 104 when the transparent substrates 101 and 102 are affixed to each other. The feeding path 104 crosses the first flow path 103a at right angles. The transparent substrates 101 and 102 are made of transparent resin emitting little self-fluorescence such as polycarbonate and formed by injection molding. The flow path 103 and the feeding path 104 may be produced by a groove formed only on either the transparent substrate 101 or the transparent substrate 102.

The cross-sectional shape of the flow path 103 perpendicular to the liquid flowing direction (the direction indicated by an arrow F in the figure) is a circular shape. The flow path 103 is sectioned in three connected paths 103a, 103b, and 103c having different cross-sectional areas, or widths. The diameters of the cross sections or widths of the flow paths 103a, 103b, and 103c are 100 μm, 300 μm, and 900 μm, respectively, as an example in this embodiment such that the width increases in the downstream direction. The shapes of the cross sections may be any shapes such as elliptical shapes other than circular shapes. The flow path 103 has linear parts and folded parts (or connecting parts). The widths of the cross-sectional areas vary at the folded parts, but the widths of each of the linear parts are constant.

The first opening 105 communicates with the upstream end of the feeding path 104, and is connected with the valve 206 and the syringe pump 201 via the silicon tube or the like. The second opening 106 communicates with the upstream end of the first flow path 103a, and is connected with the valve 207 and the syringe pump 202 via the silicon tube or the like. The third opening 107 communicates with the downstream end of the third flow path 103c as an escape path for the air when liquid flows through the flow path 103.

The method of charging reaction liquid to the biological sample reaction chip 10 is now explained.

The reaction liquid includes target nucleic acids and reagents for PCR reaction. The reagents contain predetermined concentrations of primer, polymerase, nucleotide (dNTP), and fluorescence dye SYBRGreen© suited for the biological sample reaction described later.

The target nucleic acids may be a DNA extracted from a biological sample such as blood, urine, saliva, and cerebrospinal fluid, or a cDNA produced by reversely transferring an extracted RNA.

As illustrated in FIG. 1, the container 301 filled with reaction liquid and the container 302 filled with mineral oil (liquid unmixable with the reaction liquid) are attached to the reaction liquid storage unit 205 of the biological sample quantifying device 20. Then, the syringe pump 201 and the container 301 are connected by operating the valve 206, and the syringe pump 202 and the container 302 are connected by operating the valve 207. In this condition, the reaction liquid is sucked from the container 301 into the syringe pump 201 by driving the syringe pump 201, and the mineral oil is sucked from the container 302 into the syringe pump 202 by driving the syringe pump 202.

Then, the first opening 105 is connected with the syringe pump 201 by operating the valve 206, and the second opening 106 is connected with the syringe pump 202 by operating the valve 207. In this condition, the reaction liquid is supplied from the first opening 105 into the flow path 103 by driving the syringe pump 201, and the mineral oil is supplied from the second opening 106 into the flow path 103 by driving the syringe pump 202.

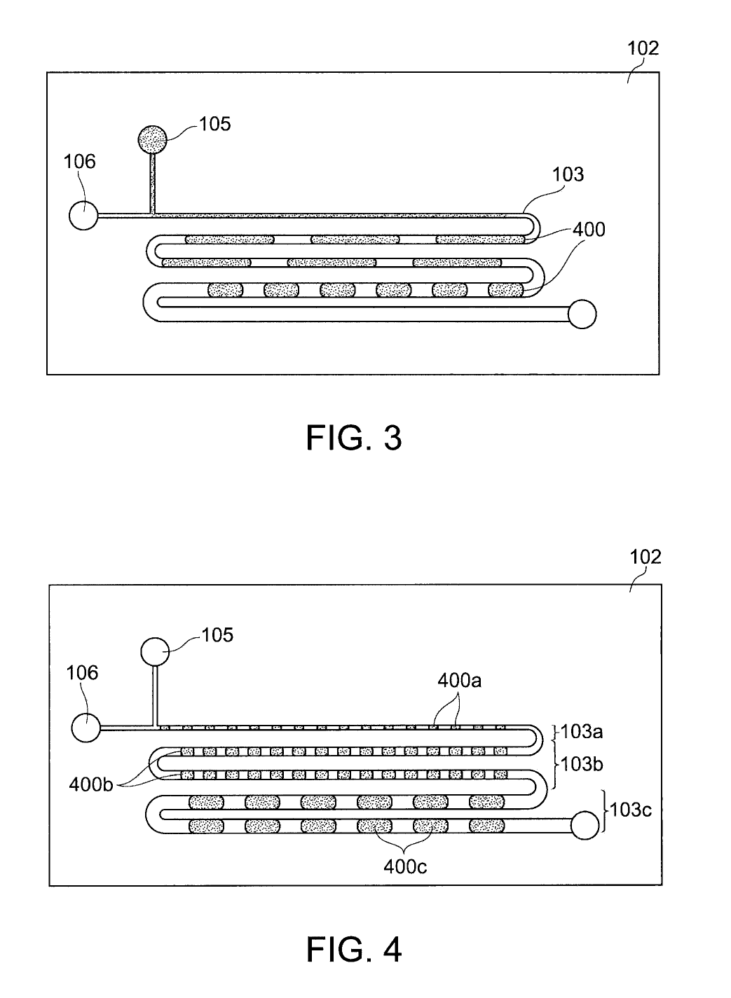

FIG. 3 illustrates a condition in which the reaction liquid and the mineral oil are charged to the biological sample reaction chip 10. As illustrated in FIG. 3, liquid masses 400 of the reaction liquid are formed within the flow path 103 in such a condition as to be separated from one another by the mineral oil by controlling the liquid feeding speeds of the syringe pumps 201 and 202 using the pump control unit 208. The liquid masses 400 contact the entire circumference of the inner wall surface of the first flow path 103. Initially, the liquid masses 400 having long and narrow shapes in the liquid feeding direction are formed in the flow path 103a having the smallest cross-sectional area, or the width. Since the cross-sectional area of the flow path 103 gradually increases in the downstream direction, the ratio of the lengths of the liquid masses 400 in the liquid feeding direction to the diameters of the liquid masses 400 gradually decreases.

The lengths of the liquid masses 400 in the longitudinal, liquid feeding direction and the spaces between the respective liquid masses 400 can be adjusted by controlling the liquid feeding speed. When the reaction liquid and the mineral oil are simultaneously supplied assuming that the liquid feeding speeds of the reaction liquid and the mineral oil are x and y, respectively, the lengths of the liquid masses 400 in the liquid feeding direction and the spaces between the liquid masses 400 are determined according to the liquid feeding speed ratio x/y. Thus, the lengths of the liquid masses 400 in the liquid feeding direction and the spaces between the liquid masses 400 can be controlled by varying the liquid feeding speed ratio of the reaction liquid to the mineral oil.

Alternatively, it is possible to keep supplying either the reaction liquid or the mineral oil at a constant speed while alternately supplying and stopping the other liquid. In this case, the lengths of the liquid masses 400 in the liquid flowing direction and the spaces between the liquid masses 400 can be controlled by changing the timing for supplying and stopping the other liquid. For example, when the mineral oil is supplied at a constant speed and the reaction liquid is alternately supplied or stopped, the spaces between the liquid masses 400 can be controlled according to the timings of starting or stopping the supply of the reaction liquid. In this case, the lengths of the liquid masses 400 in the liquid flowing direction can be controlled by varying the liquid feeding speed of the reaction liquid. The width, or the lengths of the liquid masses 400 in the liquid feeding direction become larger as the feeding speed of the reaction liquid increases.

According to this embodiment, liquid mass groups 400a, 400b, and 400c are formed in the first, second, and third flow paths respectively 103a, 103b, and 103c, as illustrated in FIG. 4, so that each liquid mass has the ratio of 1:1 in the width and in the longitudinal, liquid flowing direction. The liquid mass groups 400a, 400b, and 400c having different volumes as shown in FIG. 4 can be produced by gradually decreasing the sizes of the liquid masses 400 formed within the first flow path 103 under the control of the liquid feeding speeds of the syringe pumps 201 and 202. The liquid masses contained in a single liquid mass group are equal. Hence, the liquid masses 400a in the first flow path 103a have substantially the same volume, and the liquid masses 400b (400c) in the second flow path 103b (the third flow path 103c) have substantially the same volume. In this particular embodiment, the volume of each liquid mass contained in the liquid mass group 400b is set at 1/10 of the volume of each mass of the liquid mass group 400c, and the volume of each liquid mass contained in the liquid mass group 400a is set at 1/100 of the volume of each mass of the liquid mass group 400c.

As can be seen, a number of the masses 400 of the reaction liquid having three different volumes can be formed within the flow path 103. Thus, an operation equivalent to charging reaction liquid to plural reaction containers having three different types of volume can be performed only by operating the syringe pumps 201 and 202.

It is preferable that the ratio of the diameter of each cross section of the liquid masses 400 perpendicular to the liquid flowing direction to the mass length in the liquid feeding direction is set at 1:1. When the mass length in the longitudinal, liquid feeding direction is too long in relation to the width or the diameter of the cross section, the distribution of the target nucleic acids in the reaction liquid are too scarcely distributed for an accurate reaction or for accurately measuring the result.

After the reaction liquid is supplied to the biological sample reaction chip 10 by the above steps, a PCR process (biological sample reaction process) is executed. The PCR process is carried out within the biological sample quantifying device 20 with the first opening 105, the second opening 106, and the third opening 107 sealed. The biological sample reaction chip 10 is disposed on the heat block 203 such that reaction can repeatedly occur at predetermined temperatures in a cycle of several minutes. According to a typical method, a cycle containing a step for dissociating a double-stranded DNA at 95° C., a step for annealing primer at approximately 62° C., and a step for duplicating a complementary strand at approximately 72° C. by using heat-resistant DNA polymerase is repeated fifty times. The method of the invention can also be employed for another type of PCR in which the target DNA is heated in two-step temperature settings in a single cycle.

The mineral oil between the reaction liquid masses 400 has function for preventing evaporation of the reaction liquid and contamination by separating each reaction liquid mass 400. After the PCR process, each of the fluorescent intensities of the liquid masses 400 within the flow path 103 is measured by using an optical detector 204. If a predetermined or larger fluorescent intensity is observed in a reaction liquid mass 400, it means the reaction liquid mass 400 contains the amplified target nucleic acid. That is, the reaction liquid contains 1 or more target nucleic acids. Thus, the liquid mass group which includes both the liquid masses containing the amplified target nucleic acids and the liquid masses containing no amplified target nucleic acid is selected from the liquid mass groups 400a, 400b, and 400c having different volumes, and the number of the liquid masses 400 having no amplified target nucleic acid in the selected liquid mass group is counted to obtain the rate of the liquid masses 400 containing no target nucleic acid.

Then, the concentration of the target nucleic acids within the reaction liquid is calculated based on the above result. This concentration is calculated from the Poisson distribution. According to the Poisson distribution, the probability that a phenomenon caused with a probability p occurs x times in n trials can be calculated by the following equation:

f(x)=e⁻μμ^{x}/x! (1)

The value μ is an average, and the equation μ=np holds. When the average number of the target nucleic acids within one reaction chip is μ, the probability that the number of the target nucleic acid within the reaction chip becomes zero is expressed as follows according to the equation (1):

f(0)=e⁻μ (2)

The value f(0) corresponds to the rate of the liquid masses 400 containing no target nucleic acid calculated from the above counting result. Thus, the value μ is obtained from the equation (2), and each volume of the liquid masses is calculated from the driving conditions of the syringe pumps 201 and 202. Accordingly, the concentration of the target nucleic acids in the reaction liquid can be calculated based on these values. According to this embodiment, the liquid masses 400a, 400b, and 400c of the reaction liquid having different volumes are formed within the flow path 103 of the biological sample reaction chip 10 by controlling the liquid feeding speeds of the syringe pumps 201 and 202, and then the PCR process is performed by the biological sample reaction chip 10 at a time. Thus, quantification of nucleic acids by the critical dilution method can be efficiently carried out. In this embodiment, the volumes of the liquid mass groups 400b and 400a are 1/10 and 1/100 of that of the liquid mass group 400c, respectively. In this case, measurement equivalent to the case which uses reaction liquid diluted by 10 times and 100 times for PCR reaction can be performed. Thus, labor for diluting the reaction liquid can be eliminated. Moreover, reaction systems corresponding to arbitrary dilution magnifications can be obtained by arbitrarily changing combinations of the liquid mass group volumes. In addition, the statistical reliability can be improved by increasing the number of the liquid masses. Since the liquid mass groups 400b and 400c having relatively large volumes are formed within the second and third flow paths 103b and 103c in the downstream part of the first flow path 103a, the length of each liquid mass thus formed in the liquid feeding direction does not become extremely large in relation to the corresponding flow path cross section. As a result, the distribution of the target nucleic acids in the reaction liquid is equalized, and thus reaction variations can be reduced.

Moreover, the labor of dividing reaction liquid prior to charge by using a pipette is eliminated. Thus, even an extremely small amount of reaction liquid difficult to be distributed using a pipette can be used for reaction.

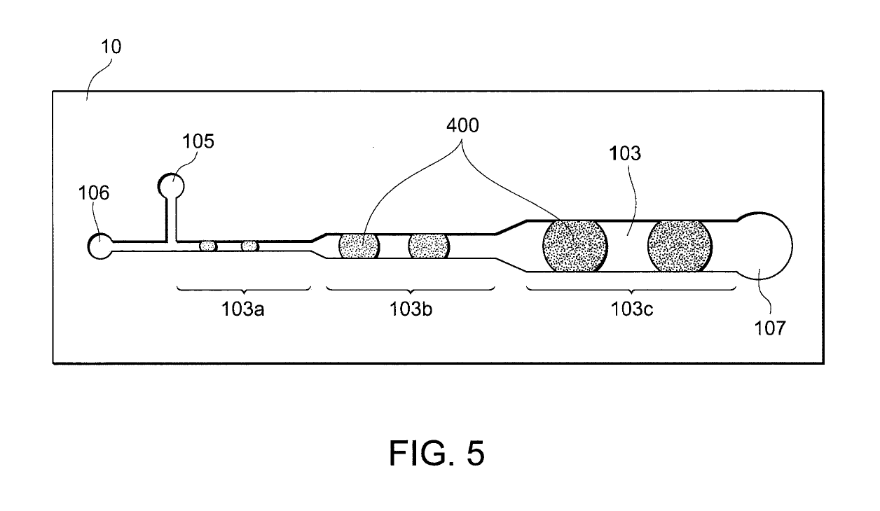

The shape of the first flow path 103 is not limited to that shown in FIG. 1 but may be other shapes as long as they include a plurality of areas having different cross-sectional areas or widths as illustrated in FIG. 5. It is preferable that the cross-sectional area gradually increases in the downstream direction. When the cross-sectional area in the upstream part is larger, small liquid masses formed in accordance with the area having the small cross-sectional area flow within the flow path having the large cross-sectional area. In this case, there is a possibility that liquid masses pass other liquid masses within the flow path.

What is claimed is:

1. A biological sample reaction chip comprising:
a first path having a first width;
a second path having a second width wider than the first width, wherein the first path and the second path are connected;
a first opening through which a first liquid is introduced into the first path; and
a second opening through which a second liquid unmixable with the first liquid is introduced into the first path.

2. A biological sample charging device comprising:
a biological sample reaction chip,
a first pump capable of supplying the first liquid into the biological sample reaction chip; and
a second pump capable of supplying the second liquid into the biological sample reaction chip,
wherein the biological sample reaction chip includes,
a first path having a first width,
a second path having a second width wider than the first width, wherein the first path and the second path are connected,
a first opening through which a first liquid is introduced into the first path, and
a second opening through which a second liquid unmixable with the first liquid is introduced into the first path.

3. The biological sample charging device according to claim 2, further comprising a pump control unit which controls at least either the liquid feeding speed of the first pump or the liquid feeding speed of the second pump.

4. A biological sample charging method comprising:
supplying a first liquid and a second liquid into a first path of a biological sample reaction chip;
forming a plurality of first liquid masses and a plurality of second liquid masses; and
controlling supply of the first liquid or the second liquid being injected,
wherein the controlling the supply causes the plurality of the first liquid masses in the second path to have larger volume than the plurality of the first liquid masses in the first path.
